(19) United States
(12) Patent Application Publication
(10) Pub. No.: US 2010/0106171 A1
(43) Pub. Date: Apr. 29, 2010

(54) TRANSCAVAL MESENTERIC VENOUS ANASTOMOSIS AND ACCESS SYSTEM

(75) Inventors: Aravind Arepally, Baltimore, MD (US); Parag V. Karmarkar, Columbia, MD (US); Qian Di, Ellicott City, MD (US); Ergin Atalar, Columbia, MD (US)

Correspondence Address: MCKENNA LONG & ALDRIDGE LLP, 1900 K STREET, NW, WASHINGTON, DC, 20006, US

(73) Assignee: JOHNS HOPKINS UNIVERSITY, Baltimore, MD (US)

(21) Appl. No.: 11/919,870

(22) PCT Filed: May 5, 2006
(86) PCT No.: PCT/US2006/017405

§ 371 Date: Jun. 30, 2008

Related U.S. Application Data
(60) Provisional application No. 60/678,339, filed on May 6, 2005.

(51) Int. Cl. A61B 17/00

(52) U.S. Cl. 606/153

(57) ABSTRACT

Disclosed is a system for creating an anastomosis between the inferior vena cava (IVC) and the mesenteric portal vein. The system includes an anastomosis device and a catheter. The anastomosis device brings the IVC and the portal vein into apposition. The resulting anastomosis enables treatment for portal hypertension as well as provides repeated and easy access to the portal system for direct delivery of therapeutic agents to portal organs. The anastomosis device has a proximal flange part, a distal flange part, a flow lumen between them. The proximal and distal flange parts include a plurality of radial struts. A valve part is disposed on one or both of the proximal and distal flange parts. The anastomosis device is made of a memory shape material. The catheter contains a collapsed anastomosis device for insertion and has one or more RF antennas to make the catheter visible under MRI guidance.

This application claims the benefit of U.S. Provisional Patent Application No. 60/678,339 filed on May 6, 2005, which is hereby incorporated by reference for all purposes as if fully set forth herein.

The research and development effort associated with the subject matter of this patent application was supported by the National Institutes of Health (NIH) under Grants NIH R01 57483 and 1K08EB004348-01.

### BACKGROUND OF THE INVENTION

1. Field of the Invention

The present invention generally relates to devices and systems for percutaneously forming an anastomosis between two vascular lumens or two anatomical chambers, wherein one of the lumens or chambers is not directly accessible by conventional percutaneous interventional procedures. More particularly, the present invention relates to devices and systems for forming an anastomosis between the inferior vena cava (IVC) and the portal vein of the mesenteric venous system, whereby the anastomosis enables periodic and relatively easy access to the mesenteric venous system.

2. Discussion of the Related Art

There are numerous health problems related to the mesenteric system that are among the leading causes of death in the United States, including diabetes, pancreatic cancer, liver cancer, and liver cirrhosis. There are many established and emerging therapies to treat these diseases.

Diabetes affects approximately 7% of the population of the United States, afflicting approximately 20.8 million children and adults. Islet cell transplantation has shown great promise for treating diabetes as an alternative to insulin injection. Islet cell transplantation involves the direct injection of islet cells into the portal vein of a patient's liver. Successful implementation of islet cell transplantation requires periodic direct access to the portal vein and liver.

Liver cancer is on the rise in the U.S. It is estimated that there will be 18,500 new cases of primary liver cancer diagnosed in the U.S. in 2006. Pancreatic cancer, the fifth leading cause of cancer death in the U.S., is diagnosed in more than 29,000 people in the U.S. every year. Successful treatment of liver and pancreatic cancers would be greatly enabled by the ability to directly introduce therapeutic agents.

Liver cirrhosis is a consequence of liver disease, in which healthy tissue is replaced by scar tissue. Cirrhosis generally leads to portal hypertension, in which liver scar tissue prevents blood flow through the liver, which in turn increases blood pressure in the portal system. Left unchecked, portal hypertension may cause abdominal swelling, damage other organs in the portal system, and may cause fatal bleeding.

Related art approaches to treating portal hypertension include the following options: liver transplantation, creation of surgical portosystemic shunts, and the creation of a transjugular intraheptic portosystemic shunt (TIPS). Because of limited availability of donor livers and technical surgical expertise, transplantation is not a viable option for the majority of candidates. Surgeries to create portosystemic shunts are invasive and generally have high complication rates. As for TIPS procedures, because TIPS is a total shunt (no flow to the portal vein), there is a higher incidence of encephalopathy, rebleeding rate, and shunt occlusion as compared to surgical shunts. Further, the TIPS procedure is generally performed without direct visualization of the portal venous system, which may incur complications such as traversal of the liver capsule and creation of fistulous tracts from the shunt to the hepatic artery of bile ducts. The inherent difficulty of creating portosystemic and TIP shunts precludes less invasive procedures, such as percutaneous procedures.

Related art treatments for liver cancer, pancreatic cancer, diabetes (via islet cell transplantation) are difficult because they require access the mesenteric venous system directly through the liver. This has risks of complications. Further, related art treatments only allow temporary access to the mesenteric system.

Accordingly, there is a need for a system for treating portal hypertension that is percutaneous, may be visualized under conventional medical imaging systems (e.g., MRI), and does not have the limitations of a total shunt. Further, there is a need for a system for creating percutaneous, periodic, and direct access to the mesenteric venous system for the delivery of therapeutic agents directly into the diseased mesenteric organ.

### SUMMARY OF THE INVENTION

The present invention provides a transcaval mesenteric venous anastomosis and access system that obviates one or more of the aforementioned problems due to the limitations of the related art. The present invention provides this by creating an anastomosis between the IVC and the portal vein, wherein the anastomosis may provide temporary and on-demand percutaneous access to the mesenteric venous system, which is otherwise not accessible by the related art percutaneous techniques.

Accordingly, one advantage of the present invention is that it enables safe and repeated access to the mesenteric system.

Another advantage of the present invention is that it provides a safe, more effective treatment for portal hypertension.

Additional advantages of the invention will be set forth in the description that follows, and in part will be apparent from the description, or may be learned by practice of the invention. The advantages of the invention will be realized and attained by the structure pointed out in the written description and claims hereof as well as the appended drawings.

To achieve these and other advantages in accordance with the present invention, a device for creating an anastomosis between two vascular lumens is provided, which comprises a first flange part; a second flange part; a flow lumen part disposed between the first flange part and the second flange part; and a valve part disposed on the first flange part.

In another aspect of the present invention, the aforementioned and other advantages are achieved by a device for creating an anastomosis between two vascular lumens, which comprises a first flange part; a second flange part; and a flow lumen part disposed between the first flange part and the second flange part, wherein the first flange part, the second flange part, and the flow lumen part include a wire having a memory shape material.

In another aspect of the present invention, the aforementioned and other advantages are achieved by a method for creating an anastomosis between a first and a second vascular lumen. The method comprises inserting a catheter into the first vascular lumen; guiding the catheter to a desired location for the anastomosis; puncturing the first vascular lumen; puncturing the second vascular lumen; inserting the catheter into the second vascular lumen; deploying a distal flange part of an anastomosis device within the second vascular lumen; bringing the second vascular lumen into apposition with the first vascular lumen; and deploying a proximal flange part of the anastomosis device within the first vascular lumen.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1A illustrates an exemplary anastomosis device according to a first embodiment of the present invention;

FIG. 1B is a plan view of the exemplary anastomosis device of FIG. 1A;

FIG. 1C illustrates a wire structure of the exemplary anastomosis device of FIG. 1A;

FIG. 1D illustrates exemplary dimensions corresponding to the wire structure of FIG. 1C;

FIG. 1E illustrates an exemplary anastomosis device having a plurality of capacitors;

FIG. 1F illustrates a wire structure of the exemplary anastomosis device of FIG. 1E;

FIG. 2A illustrates an exemplary anastomosis device according to a second embodiment of the present invention;

FIG. 2B is a plan view of the exemplary anastomosis device of FIG. 2A;

FIG. 3A is a cutaway view of an exemplary insertion catheter according to the present invention;

FIG. 3B illustrates an exemplary insertion catheter having RF antennas for visualization under MRI;

FIG. 4 illustrates an exemplary process for deploying the anastomosis device according to the present invention; and

FIGS. 5A-E illustrate an exemplary process for inserting and deploying an anastomosis device according to the present invention.

### DETAILED DESCRIPTION OF THE ILLUSTRATED EMBODIMENTS

The present invention involves a device for percutaneously bringing two vascular lumens into apposition and creating an anastomosis between the two vascular lumens. The anastomosis device may enable flow between the two vascular lumens, or the anastomosis device may keep the two vascular lumens isolated from each other to maintain the naturally present conditions. The anastomosis device provides an access point for repeated percutaneous access to an otherwise isolated or inaccessible vascular lumen. The anastomosis device provides a safe access point for advancing interventional devices, under x-ray, MRI or CT imaging guidance, for the purpose of delivering, for example, a therapeutic or a diagnostic device to the vascular lumen. For purposes herein, the term “vascular lumen” may refer an artery, a vein, an anatomical chamber, or the like.

The anastomosis device may have a valve structure for nominally sealing the anastomosis created by the device, which may enable periodic percutaneous access between the two vascular lumens. The valve structure on the anastomosis device may be nominally closed so that the IVC and the mesenteric portal vein may be nominally isolated. The valve structure has sufficient flexibility to enable a needle or an interventional device to pass through the anastomosis for the purposes of injecting therapeutic agents directly into the organ being treated (e.g., the liver or the pancreas). The valve structure on the anastomosis device may have a predetermined rigidity such that, if a pressure differential between the IVC and the mesenteric portal vein increases beyond a certain threshold, the valve will temporarily open to alleviate the pressure differential. For patients with portal hypertension, the valve structure enables the routing of blood from the mesenteric system into the IVC, thus decompressing the portal system and maintaining a naturally occurring pressure gradient between the two vessels. In doing so, blood is routed through the liver, thus utilizing the functionality of the liver. In an alternate embodiment, the valve structure may not be present.

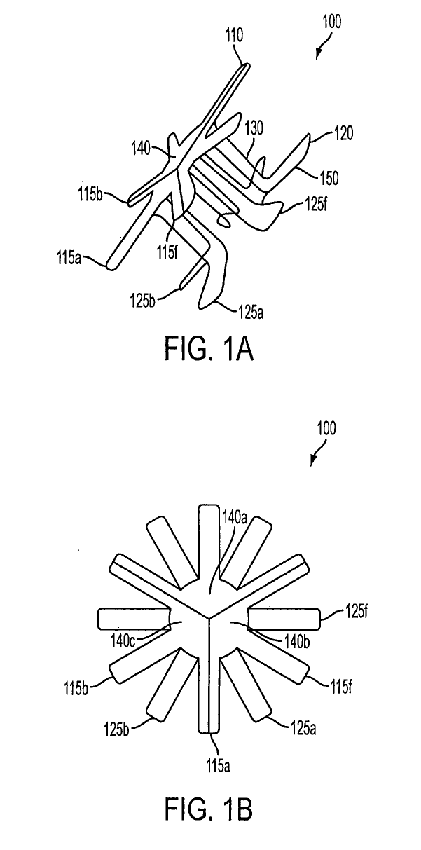

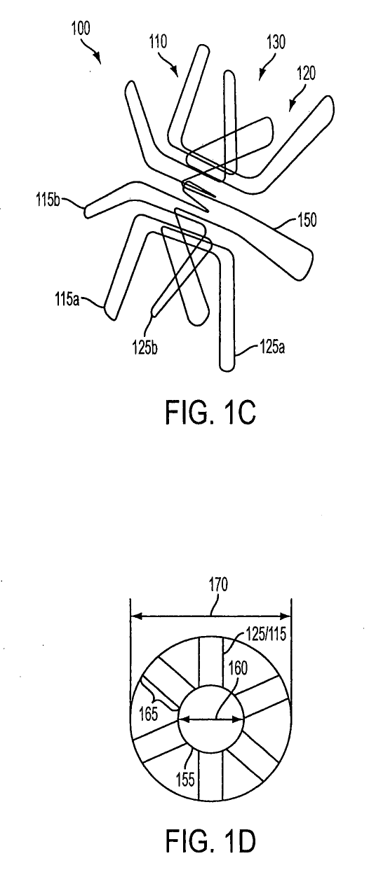

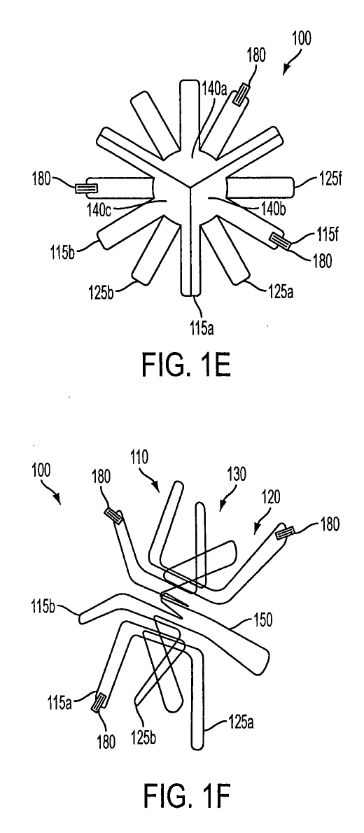

FIG. 1A illustrates an anastomosis device 100 according to a first embodiment of the present invention. Anastmosis device 100 includes a proximal flange part 110, a distal flange part 120, and a flow lumen part 130 between proximal flange part 110 and distal flange part 120. Proximal flange part 110 has a plurality of proximal radial struts 115a-f, and distal flange part 120 has a plurality of distal radial struts 125a-f. Disposed on the plurality of proximal radial struts 115a-f is a valve part 140, which may include a plurality of leaflet parts 140a-c.

Proximal flange part 110 and distal flange part 120 are used to hold the vascular lumens apposed to each other, as illustrated in greater detail below. Flow lumen part 130 provides a flow conduit for blood (or other fluids) and provides a path for a surgeon to advance an interventional diagnostic or therapeutic device.

Proximal radial struts 115a-f define an aperture (not shown) in a center portion of proximal flange part 110. Distal radial struts 125a-f define a similar aperture in a center portion of distal flange part 120. Flow lumen part 130 defines a cylinder-like path corresponding to the respective apertures of proximal flange part 110 and distal flange part 120.

Proximal radial struts 115a-f, distal radial struts 125a-f, and flow lumen part 130 may be formed by a wire 150. Wire 150 may include a memory shape material, such as Nitinol. For purposes of illustration and not limitation, the term “wire” will be used, but it will be readily apparent to one skilled in the art that “wire” may refer to a plurality of wires. Wire 150 may have a substantially cylindrical cross section, or may have a cross section of different shapes. Wire 150 may have a diameter of about 0.009″. However, one skilled in the art will readily appreciate that different diameters of wire 150 are possible and within the scope of the invention, provided that anastomosis device 100 provides sufficient mechanical holding force to maintain the two vascular lumens in apposition.

FIG. 1C illustrates exemplary anastomosis device 100, in which the valve part 140 is not present. As illustrated in FIG. 1C, the wire structure of anastomosis device 100 may include a single strand of wire 150, which is manipulated to form proximal radial struts 115a-f, distal radial struts 125a-f, and flow lumen part 130.

FIG. 1D is a plan view of the wire structure of FIG. 1C. The outer and inner circles of FIG. 1D are for illustrating diameters and are not necessarily part of the wire structure of anastomosis device 100. As illustrated in FIG. 1D, aperture 155 may have a diameter 160 of about 6 mm. Proximal and distal radial struts 115a-f and 125a-f may have a length 165 of about 6 mm. The outer diameter 170 of the Proximal and distal flange parts 110 and 120 may be about 18 mm, and may be in a range between about 1 cm to about 3 cm. These dimensions need not be the same for the proximal and distal components of anastomosis device; the proximal flange part 110 and the distal flange part 120 may have different dimensions, depending on the vascular lumens in which the anastomosis is to be formed.

Flow lumen part 130 may have a length 175 of about 3 mm. Other values for length 175 are possible and within the scope of the invention, depending on the thicknesses of the walls of the vascular lumens in which the anastomosis is to be formed. Flow lumen part 130 may have a substantially straight cylindrical shape, as illustrated in FIG. 1C, or it may have a substantially toroidal shape such that wire 150 forming flow lumen part 130 may have a substantially continuous curvature between proximal flange part 110 and distal flange part 120. Generally, a substantially toroidal curvature in flow lumen part 130 may help maintain pressure between the two vascular lumens in which the anastomosis is to be formed. In doing so, the two vascular lumens may be kept in apposition.

The first embodiment of the present invention may include six proximal radial struts 115a-f and six distal radial struts 125a-f. A greater or lesser number of radial struts are possible and within the scope of the invention.

The preferred number of radial struts, the length 165 of the radial struts 115a-f and 125a-f, the diameter 160 of aperture 155, and the length 175 of flow lumen part 130 may vary. These parameter may depend on factors such as the diameter of the anastomosis to be formed, the required stability of the anastomosis, the rigidity, strength, and the thickness of the walls of the vascular lumens in which the anastomosis is to be formed.

Referring to FIG. 1B, valve part 140 may have leaflets 140a-c, as stated above. Leaflets 140a-c may be adhered to wire 150 of proximal radial struts 115a-f. Leaflets 140a-c may be made of teflon, dacron, PVDF (polyvinylidine diflouride), PVA (polyvinyl acetate), an ultra thin membrane of Nitinol, or the like. Leaflets 140a-c may have a thickness sufficient to prevent flow through flow lumen part 130 when the pressure differential between the two vascular lumens, in which the anastomosis is formed, is below a specified pressure differential. If the anastomosis is to be formed to provide access to the portal system organs for the injection of therapeutic agents, then valve leaflets 140a-c should have a sufficient thickness so that the valve is sufficiently strong to prevent flow through the anastomosis under nominal pressure differential conditions. However, valve leaflets 140a-c should not be so thick that it would make it difficult to penetrate valve part 140 with a diagnostic or therapeutic device from the IVC. The desired thickness may depend on the material in leaflets 140a-c. Although FIG. 1A illustrates valve part 140 disposed on proximal flange part 110, one skilled in the art will realize that valve part 140 may be formed alternatively, or additionally, on distal flange part 120. Whether valve part 140 is to be disposed on proximal flange part 110, distal flange part 120, or both, will depend on the intended use of anastomosis device 110, e.g., alleviation of portal hypertension, delivery of therapeutic agents, etc.

Although FIG. 1B illustrates leaflets 140a-c as being three separate pieces of material, one skilled in the art will readily appreciate that the leaflets 140a-c may be formed from one piece of material. If one piece of material is used, then leaflets 140a-c may be created by, cutting one or more slits in the material to form a valve. Further, although FIG. 1B illustrates three leaflets 140a-c, a greater number or lesser number of leaflets may be used to form valve part 140. Factors to consider in when varying the design of valve part 140 include the strength of the valve part 140, the ease with which valve part 140 is to be penetrated for injecting therapeutic agents, and how easily valve part 140 should collapse so that it can be inserted into a catheter (discussed below).

FIGS. 1E and 1F illustrate a variation of anastomosis device 100, which has a plurality of capacitors 180. Capacitors 180 may have a capacitance to tune the structure of wire 150 to the larmor frequency of MRI (1 or more field strengths). In doing so, anastomosis device 100 may function as an RF antenna, in which anastomosis device 100 inductively couples to the surface RF transmit and receive coils used in MRI. This may make anastomosis device 100 visible in MR imagery, which may enable MRI guided implantation and monitoring of anastomosis device 100.

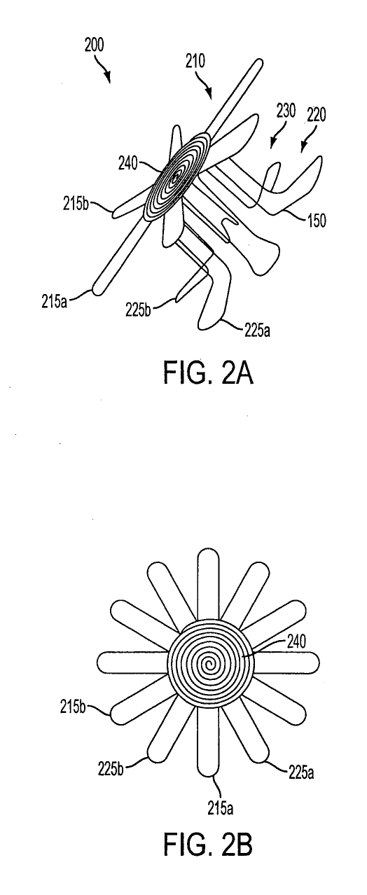

FIGS. 2A and 2B illustrate an exemplary anastomosis device 200 according to a second embodiment of the present invention. Exemplary anastomosis device 200 may be substantially similar to anastomosis device 100, but with a different valve part 240. Anastomosis device 200 may also have a plurality of capacitors (not shown) substantially similar to capacitors 180 of anastomosis device 100. Valve part 240 includes a spiral configuration of wire, such as Nitinol. More specifically, valve part 240 may have a superelastic coil that forms a seal. Valve part 240 is preferably disposed on the proximal flange part 210, the distal flange part 220, or both, as explained above.

If valve part 240 is disposed on proximal flange part 210, the proximal radial struts 215a-f may have a covering substantially similar to leaflets 140a-c of the first embodiment. The covering is to prevent leakage around valve part 240. Similarly, if valve part 240 is disposed on distal flange part 220, then distal radial struts 225a-f may have a covering.

For both embodiments, valve part 140/240 may be disposed within flow lumen part 130/230. In this case, valve part 140/240 may be attached to the radial struts of the proximal flange part 110/210 and the distal flange part 120/220.

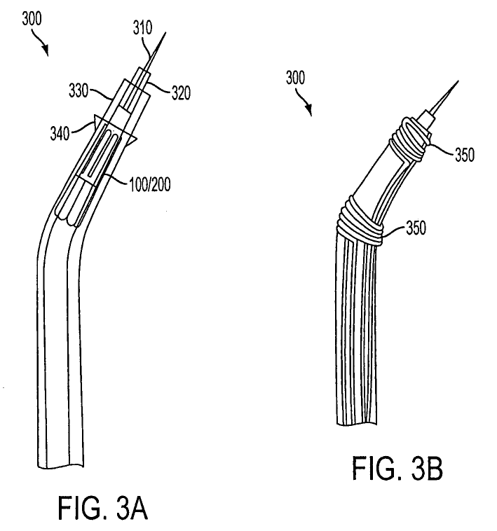

FIG. 3A is a cutaway view of an exemplary catheter 300 for deploying anastomosis device 100/200 according to the present invention. Catheter 300 preferably includes the following: a needle 310; an inner sheath 320 substantially covering needle 310; an outer sheath 330, which encompasses a folded anastomosis device 100/200; and an outer flange part 340.

Inner sheath 320 serves as a lumen for needle 310. It may also serve as a guide wire for catheter 300.

Outer sheath 330 contains anastomosis device 100/200 in a folded or collapsed configuration to minimize its diameter for delivery through the walls of the vascular lumens in which it will be deployed (described below). Outer sheath 330 may be a sheath that an operator can gradually pull to separate it from anastomosis device 100/200 to deploy the device. Alternatively, outer sheath 330 may include a fiber weave crimping/delivery device, which has a knit cord encasing anastomosis device 100/200. The knit cord may be unraveled by the operator to deploy anastomosis device 100/200.

Outer flange part 340 may have one or more radial protrusions for preventing separation of the two vascular lumens during deployment of anastomosis device 100/200. This will be described further below.

FIG. 3B illustrates an exemplary catheter 300 having one or more radio frequency (RF) antennas 350 disposed at its tip and along its length. RF antennas 350 provide for visibility under MRI and thus enable deployment of the anastomosis device using MRI guidance. RF antennas 350 include tuning circuitry (not shown) to match their respective resonant frequencies of the MRI larmor frequency. In doing so, catheter 300 may be visible along its length under MRI, which will provide MR imagery-based guidance to the surgeon deploying anastomosis device 100/200 according to the present invention. A description of the RF antennas 350 is disclosed in International Patent Application No. PCT/US2005/017351, Publication No. WO 2005/112836 A2, titled INTERVENTIONAL DEVICES FOR CHRONIC TOTAL OCCLUSION RECANALIZATION UNDER MRI GUIDANCE, which is hereby incorporated by reference as if fully disclosed herein.

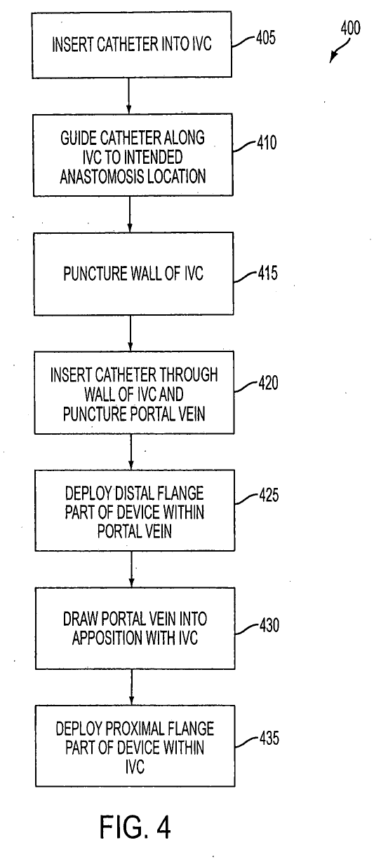

FIG. 4 illustrates an exemplary process 400 for deploying anastomosis device 100/200 according to the present invention. The description below pertains to creating an anastomosis between the IVC 510 and the mesenteric portal vein 520. However, it will be readily apparent to one of ordinary skill that process 400, along with catheter 300 and anastomosis device 100/200, may be modified to be suitable for other parts of the anatomy e.g. as an atrial septal closure device to be implanted at the patent fossa ovalis. Further, process 400, catheter 300, and anastomosis device 100/200 could be modified to perform other procedures, such as Pulmonary Artery to Subclavian Artery bypass for congenital heart disease; Pulmonary Artery to Aorta bypass for congenital heart disease; Inferior Vena Cava to Splenic Vein for Spleno-Renal Shunt for portal hypertension and venous therapies to the spleen; and Renal vein to Splenic Vein for portal hypertension and venous therapies to the spleen. All such variations are possible and within the scope of the invention.

In steps 405 and 410, catheter 300 is inserted into IVC 510 and guided to the location where an anastomosis is to be formed.

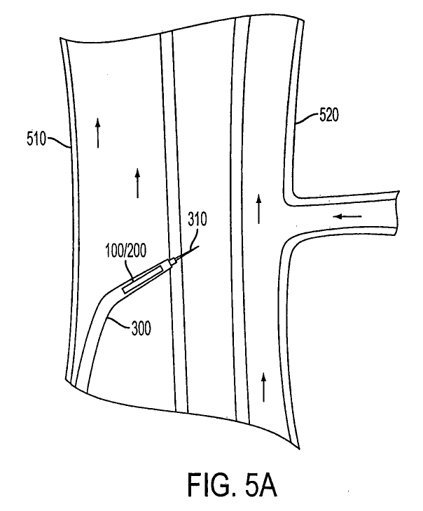

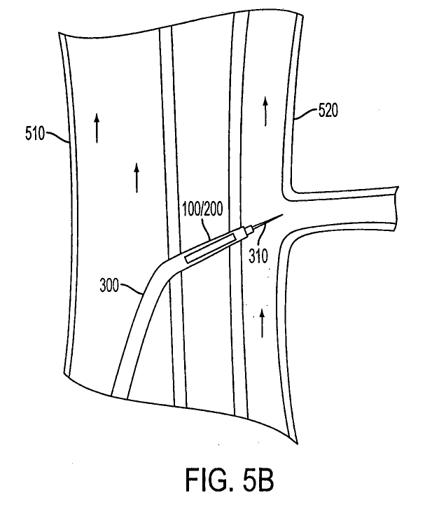

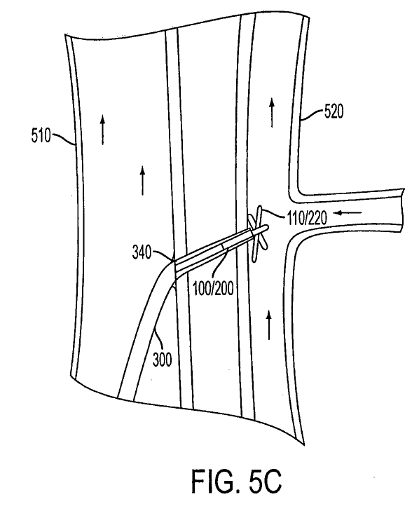

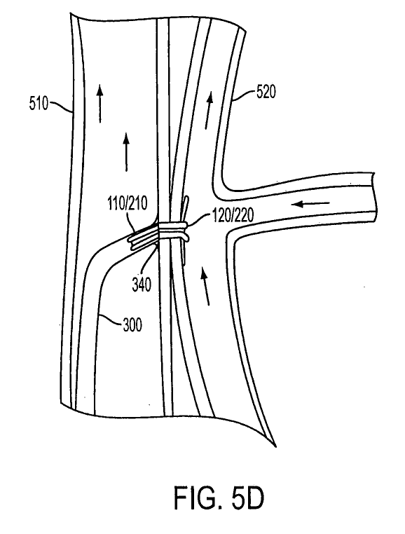

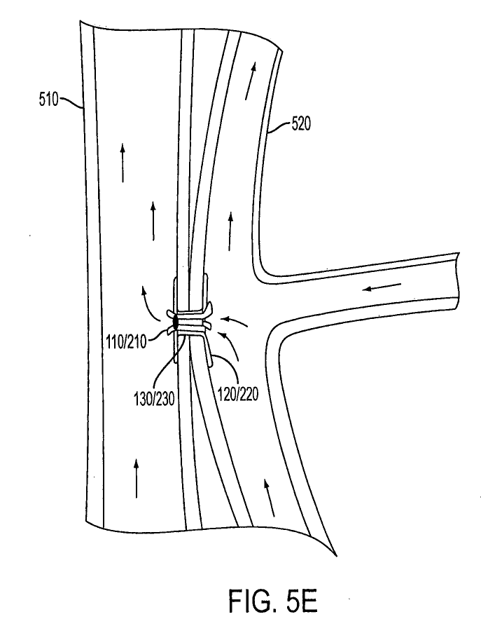

Referring to FIG. 5A, in step 415, the wall of NC 510 is punctured with needle 310 of catheter 300.

Referring to FIG. 5B, in step 420, catheter 300 is inserted through the wall of IVC 510, and then the wall of portal vein 520 is punctured by needle 310.

Referring to FIG. 5C, in step 425, distal flange part 120/220 is deployed within portal vein 520. This can be done several ways. According to one way, once the distal end of folded anastomosis device 100/200 is within portal vein 520, outer sheath 330 of catheter 300 is pulled back until distal flange part 120/220 is exposed. Being made of a shape memory material, distal flange part 120/220 opens to take its shape as illustrated in FIGS. 1A and 2A. Alternatively, if outer sheath 330 has a fiber weave crimping/delivery device, a line of the fiber weave device is pulled to unravel the delivery device. As the delivery device unravels, distal flange part 120/220 opens up to take its shape illustrated in FIGS. 1A and 2A.

Referring to FIG. 5D, in step 430, with the distal flange part 120/220 engaged against the wall of portal vein 520, catheter 300 is pulled back to bring portal vein 520 into apposition with IVC 510. In doing so, outer flange part 340 is held against the wall of IVC 510 to prevent NC 510 from retreating from portal vein 520 as catheter 300 is drawn back. Catheter 300 may include other mechanisms (not shown), such as an inflatable balloon, a collapsible stent, or an additional sheath. These additional mechanisms may further enable apposition of IVC 510 and portal vein 520 in step 430. Any of these additional mechanisms may be used to help push portal vein 520 toward IVC 510.

Referring to FIG. 5E, in step 435, proximal flange part 110/210 of anastomosis device 100/200 is deployed within NC 510. In doing so, outer sheath 330 is pulled back to expose proximal flange part 110/210. Alternatively, if outer sheath 330 has a fiber weave crimping/delivery device, the line of the fiber weave is pulled further to unravel the delivery device and expose proximal flange part 110/210. Once proximal flange part 110/210 is exposed, it opens (in a manner similar to distal flange part 120/220) to take its shape as illustrated in FIGS. 1A and 2A. Once deployed, proximal flange part 110/210 engages the wall of IVC 510 and holds it in apposition to the wall of portal vein 520.

At the conclusion of exemplary process 400, an anastomosis is created by anastomosis device 100/200 between IVC 510 and portal vein 520. Referring to FIG. 5E, the anastomosis is illustrated as being used for alleviating portal hypertension.

Anastomosis device 100/200, having valve part 140/240, enables safer and repeated access to the mesenteric system via the IVC to deliver diagnostic and therapeutic devices, drugs, cellular therapies to mesenteric system organs, such as the liver, pancreas, and spleen.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. device for creating an anastomosis between two vascular lumens, comprising:
a first flange part;
a second flange part;
a flow lumen part disposed between the first flange part and the second flange part; and
a valve part coupled to one of the first flange part, the second flange part, and the flow lumen part.

2. The device of claim 1, wherein the valve part is configured to be penetrated by an interventional device after the device is deployed.

3. The device of claim 1, wherein the valve part is configured to maintain a specified pressure differential between a first pressure at the first flange part and a second pressure at the second flange part, and wherein the valve part is configured to open if a difference between the first pressure and the second pressure is greater than the specified pressure differential.

4. The device of claim 1, wherein the first flange part comprises a plurality of radial struts.

5. The device of claim 4, wherein the first flange part comprises six first radial struts.

6. The device of claim 4, wherein the second flange part comprises a plurality of radial struts.

7. The device of claim 6, wherein the second flange part comprises six second radial struts.

8. The device of claim 1, wherein the first flange part, the second flange part, and the flow lumen part include a memory shape alloy.

9. The device of claim 8, wherein the memory shape alloy includes Nitinol.

10. The device of claim 1, wherein the valve part comprises a plurality of leaflet parts.

11. The device of claim 10, wherein the valve part comprises three leaflet parts.

12. The device of claim 1, wherein the valve part comprises a coiled wire.

13. The device of claim 12, wherein the coiled wire comprises Nitinol.

14. The device of claim 1, wherein the device is configured to be disposed between an inner sheath and an outer sheath of a catheter.

15. A device for creating an anastomosis between two vascular lumens, comprising:
a first flange part;
a second flange part; a flow lumen part disposed between the first flange part and the second flange part, wherein the first flange part, the second flange part, and the flow lumen part are formed from wire having a memory shape material; and
a valve part coupled to one of the first flange part, the second flange part, and the flow lumen part.

16. The device of claim 15, wherein the wire comprises Nitinol.

17. The device of claim 15, wherein the wire comprises a plurality of capacitors.

18. A method for creating an anastomosis between a first and a second vascular lumen, comprising:
inserting a catheter into the first vascular lumen;
guiding the catheter to a desired location for the anastomosis;
puncturing the wall of the first vascular lumen;
puncturing the wall of the second vascular lumen;
inserting the catheter into the second vascular lumen;
deploying a distal flange part of an anastomosis device within the second vascular lumen;
bringing the second vascular lumen into apposition with the first vascular lumen;
deploying a proximal flange part of the anastomosis device within the first vascular lumen; and
sealing the anastomosis by closing a valve coupled to the anastomosis device.

19. The method of claim 18, wherein the first vascular lumen is associated with the inferior vena cava.

20. The method of claim 19, wherein the second vascular lumen is associated with the mesenteric portal vein.

21. The method of claim 18, wherein deploying the distal flange part of the anastomosis device comprises pulling a sheath to expose the distal flange part within the second vascular lumen.

22. The method of claim 18, wherein the valve is configured to open if a pressure differential between the first vascular lumen and the second vascular lumen is greater than a specified pressure differential.

23. The method of claim 18, wherein the valve is configured to allow an interventional device to be inserted from the first vascular lumen, through the valve, and into the second vascular lumen.
